(19) United States
(12) Patent Application Publication
(10) Pub. No.: US 2022/0215949 A1
(43) Pub. Date: Jul. 7, 2022

(54) MEDICAL REMOTE DATA MONITORING AND FEEDBACK FOR IMPROVED OUTCOMES

(71) Applicant: Joseph Todd Piatnik, Bethel, CT (US)

(72) Inventor: Joseph Todd Piatnik, Bethel, CT (US)

(21) Appl. No.: 17/500,909

(22) Filed: Oct. 13, 2021

Related U.S. Application Data
(60) Provisional application No. 63/091,284, filed on Oct. 13, 2020.

(51) Int. Cl. G16H 40/67; A61B 5/00; G16H 10/60; G16H 80/00; G16H 20/40

(52) U.S. Cl. CPC G16H 40/67; A61B 5/0022; G16H 20/40; G16H 80/00; G16H 10/60

(57) ABSTRACT

A computer method to improve medical outcomes that receives at a backend server notification of surgery including a recovery location. Creating a plan to deliver a smart treatment device with a unique identifier to the recovery location. Associate the unique identifier to the surgery. Receiving information that the smart treatment device is at the recovery location. The smart treatment device gathers recovery data. Sending the recovery data with the unique identifier to the backend server. Receiving at the backend server the recovery data. Receiving a request for access to the recovery data for the surgery. Providing access to the recovery data. Receiving and implementing adjustments to a treatment plan. Receiving final medical outcome information. Recording the surgery, recovery data and final medical outcome in a historical database. Training a machine learning algorithm on the historical data. Using the machine learning algorithm to suggest adjustments the treatment plan.

### TECHNICAL FIELD

The present invention relates generally to equipment and system for monitoring and improving the medical treatment of a patient, and more particularly, to a system for remote data monitoring the medical treatment of a medical patient after an intervention for example like surgery.

### BACKGROUND

Currently, medical patients are sent home with a treatment plan that, if followed, will improve or, in some cases, be critical to good medical outcomes. Sometimes the treatment plan involves physical devices or movement instructions, and the patient's compliance with the treatment plan has a significant impact on outcome of the patient's successful recovery. For example, for certain surgery like knee surgery, using a Deep Vein Thrombosis (DVT) Prophylactic treatment device in the recovery treatment plan is very important and has a big influence on the success and speed of recovery. Currently, the patient will have to follow up visits with their doctor, at which point the doctor will ask the patient about their use of the device, and the doctor will examine how the recovery is going. At this point, the doctor can determine if any adjustments to the treatment plan are appropriate. This adjustment is typically days or weeks later, by which time the optimization of the healing of the damaged area, may have passed. For example, if the damage area is a joint then for those injured for the cells to proper differentiation into either sliding surfaces or strong support surfaces may have passed.

What is needed is a system that allows the doctor to know earlier if the patient is complying with the treatment plan earlier so proper adjustments can be determined and better outcomes can be pursued.

### SUMMARY OF THE INVENTION

A method to improve medical outcomes implemented on an electronic computer that includes receiving at a backend server notification of surgery including a recovery location. Creating a plan deliver a smart treatment device to the recovery location. The smart treatment device has a unique identifier. Associate the unique identifier to the surgery. Receiving information that the smart treatment device at the recovery location. Gathering recovery data by the smart treatment device. Sending the recovery data with the unique identifier to the backend server. Receiving at the backend server the recovery data. Receiving a request for access to the recovery data for the surgery. Providing access to the recovery data. Receiving adjustments to a treatment plan. Implementing adjustments to the treatment plan. Receiving final medical outcome information. Recording the surgery, recovery data and final medical outcome in a historical database. Training a machine learning algorithm on the historical data. Using the machine learning algorithm to suggest adjustments the treatment plan.

The medical remote monitoring system also includes a backend server with a medical provider role with rights to access a patient identifier and that patient identifier associated recovery data. The system also includes a treatment device with a treatment modality that is performed based on commands received and record usage information about the treatment modality where the treatment device communicates the recovery data associated with a unique identifier to the backend server. The system also includes a mobile device executing a software application configured to provide the commands. The system also includes the backend server uses the usage information to for display over a user interface accessible having authorized access to the patient identifier.

The system where the treatment modality includes mechanical deep vein thrombosis prophylaxis. The system where the usage information includes details related to schedule, duration, and usage of the treatment device. The system where the recovery data includes data related to usage of the treatment device, physiological data, and data entered into the software application in response to questions presented to the patient on the software application. The system where the unique identifier is the treatment device's identifier. The system where the treatment device's identifier is associated with a surgery id, and where the surgery id is associated with the patient identifier. The system where the treatment device is a prophylactic treatment device. The system where the backend server provides access to the recovery data and receives interventions for improving health of the patient. The system where the interventions are obtained from a machine learning model pre-trained on health improvement data of patients and their outcomes.

A method for improving treatment outcomes. The method also includes collecting recovery data of a patient which includes usage information obtained from a durable medical equipment treatment device, physiological data obtained from sensors, and patient entered data. The method also includes associating the recovery data with a unique identifier. The method also includes encrypting the recovery data associated with the unique identifier. The method also includes transmitting the encrypted data to a backend server. The method also includes decrypting the encrypted data at the backend server to obtain the health data and unique identifier. The method also includes providing access of the recovery data to enable remote data monitoring by a medical provider.

The method where the recovery data is gathered using one or more of an oximeter, glucose monitor, and weight scale. The method where the usage information includes details related to duration and usage of the treatment device. The method where the unique identifier is the treatment device's identifier. The method where the treatment device's identifier is associated with a surgery id, and where the surgery id is associated with a patient identifier. The method may include storing previous patient data collected about treatment data, adjustment to a treatment plan for the patient, and final outcomes for the patient. The method may include training a machine learning model on the previous patient data collected about treatment data, adjustment to a treatment plan for the patient, and final outcomes for the patient. The method may include receiving a suggested adjustment to the treatment plan from the machine learning model.

### BRIEF DESCRIPTION OF THE DRAWING

Reference numerals may be repeated among the figures to indicate corresponding or analogous elements. The figures are listed below.

FIG. 1 is a block diagram of a system for monitoring and improving the treatment of a medical patient.

FIG. 1B is a swim lane diagram showing how the recovery data is made available to a medical practitioner.

FIG. 2 is a block diagram of the recovery location portion of the system.

FIG. 2B is a block diagram of the durable medical Equipment (DME) smart treatment/monitoring device.

FIG. 4 is a block diagram of a wireless control device that may be used in the recovery location.

FIG. 5A is a block diagram of the backend portion of the system

FIG. 5B is a block diagram of a backend portion of the system that includes machine learning recommended interventions.

FIG. 6 is a block diagram of a medical provider access to a remote treatment management system.

FIG. 7A illustrates the use of a deep vein thrombosis compression system DME treatment/monitoring device.

FIG. 7B illustrates the use of an elbow range of motion brace DME treatment/monitoring device.

FIG. 7C illustrates the use of a knee range of motion braces DME treatment/monitoring device.

FIG. 7D illustrates the use of a shoulder continuous passive motion device DME treatment device.

FIG. 7E illustrates the use of a knee continuous passive motion devices DME treatment/monitoring device.

FIG. 8 illustrates a Medical Treatment Device controller.

FIG. 9 is an overview of a wireless remote treatment management system including Patient Data and Machine Data.

FIG. 10 shows screens for a controller App.

FIG. 11 is a box diagram of a computing device.

FIG. 12A shows the left side of a screen from the backend system showing device ID (“Machine Deployed 1”).

FIG. 12B show the right side of the screen from the backend system.

FIG. 12C shows a screen displaying gathered recovery data.

FIG. 12D shows a screen from the backend system showing surgery ID.

FIG. 13 is an illustration of a flow chart of a method to provide an adjustment to a treatment plan.

FIG. 14 is an illustration of a flow chart of a method to train a machine learning module based on historical data.

FIG. 15 is an illustration of a flow chart of a method to provide an adjustment to a treatment plan based on data received from smart DME treatment device.

FIG. 16 is an illustration of a flow chart of a method to monitor a treatment plan based on data received from the smart DME treatment device.

### DETAILED DESCRIPTION

Numerous specific details are set forth in order to aid in developing a thorough understanding. However, it will be understood that in practice the functionality or capability may be practiced without the specific details presented. In other instances, well-known methods, procedures, components, units, and circuits are not described in detail so as not to obscure the discussion.

The term “wireless device,” as used in this document, includes, for example, a device capable of wireless communication, a communication device capable of wireless communication, a communication station capable of wireless communication, a portable or non-portable device capable of wireless communication, or the like. The wireless device may be or may include a peripheral that is integrated with a computer, or a peripheral that is attached to a computer. The term “wireless device” may include a wireless service.

The term “communicating” as used in this document with respect to a communication signal may include transmitting the communication signal or receiving the communication signal. For example, a communication unit, which is capable of communicating a communication signal, may include a transmitter to transmit the communication signal to another communication unit and a communication receiver to receive the communication signal from another communication unit. The verb communicating may be used to refer to the action of transmitting or the action of receiving. For example, the phrase “communicating a signal” may refer to the action of transmitting the signal by a first device, and may not necessarily include the action of receiving the signal by a second device. In another example, the phrase “communicating a signal” may refer to the action of receiving the signal by a first device, and may not necessarily include the action of transmitting the signal by a second device. The communication signal may be transmitted or received, for example, in the form of Radio Frequency (RF) communication signals, or in the form of wired communication signal or any other type of signal.

As used in this document, the term “circuitry” may include, an Application Specific Integrated Circuit (ASIC), an integrated circuit, an electronic circuit, a processor (shared, dedicated, or group), memory (shared, dedicated, or group), that execute one or more software or firmware programs, a combinational logic circuit, or other suitable hardware components that provide the described functionality. Circuitry may be implemented in, or functions associated with the circuitry may be implemented by one or more software or firmware modules. Circuitry may include logic, at least partially operable in hardware.

The term “logic” may refer to computing logic embedded in the circuitry of a computing apparatus, computing logic stored in memory of a computing apparatus. For example, the logic may be accessible by a processor of the computing apparatus to execute computing logic to perform computing operations. Logic may be embedded in various types of memory or firmware, e.g., silicon blocks of various chips or processors. Logic may use various circuitry, e.g., radio circuitry, receiver circuitry, control circuitry, transmitter circuitry, transceiver circuitry, processor circuitry, and the like. Logic may use volatile memory or non-volatile memory, including random access memory, read-only memory, programmable memory, magnetic memory, flash memory, persistent memory, and the like. Logic may be executed on one or more processors using memory, e.g., registers, stack, buffers, or the like, coupled to the one or more processors, e.g., as necessary to execute the logic.

The system may use a WLAN, e.g., a WiFi network or with any other suitable wireless communication network, for example, Bluetooth, Bluetooth low energy (BLE), and the like.

The term “antenna,” as used in this document, may include any suitable configuration, structure, or arrangement of one or more antenna elements, components, units, assemblies or arrays. The antenna may implement transmit and receive functionalities using separate transmit and receive antenna elements. The antenna may implement transmit and receive functionalities using common or integrated transmit/receive elements. The antenna may include, a phased array antenna, a single element antenna, a set of switched beam antennas, or the like.

The term “module,” as used in this document, may be an object file that contains code that runs on the kernel environment. For example, the module may be a part of a computer program. Programs may be composed of one or more modules that are not combined until the program is linked. A single module may contain one or several routines. Furthermore, when the term module is used in hardware, the module is defined as a self-contained component.

The term “portable electronic device” portable electronic device, e.g., cellphone, tablet.

The term “machine learning” generally refers to computer algorithms that improve automatically through experience and includes a number of approaches and may include both supervised and unsupervised learning and reinforcement learning, to name a few.

Supervised learning needs training data, and may be from older from simpler forms of support vector machines, linear regression, logistic regression, naive Bayes, linear discriminant analysis, decision trees, to more complex k-nearest neighbor algorithm, neural networks, and other means. In general the training for supervised learning needs training data that includes a number of features (a vector of features) and the desired outcome (label). For this application the vector of features may be the treatment plan, the gathered recovery data, and treatment plan adjustments. The desired outcome (label) may be the final outcome (good/bad) time for recovery (1 week, 2 weeks, 3 weeks . . . ), time to return to work (1 day, 2 days, 3 days), reported happiness of the patient with the outcome, financial cost for good outcome. Using supervised learning the training data can develop an algorithm that can have a high accuracy of predicting the final outcome. Using this algorithm various interventions can be tested against the algorithm to see which one improves the odds of the desired final outcome.

A smart controller may connect a simple device to a backend system that may include machine learning capabilities. For example, the machine learning capabilities may have trained an algorithm that may control the simple device, e.g., a DME treatment device, to operate according to instructions be provided by the algorithm (for example, an artificial neural network, a deep neural network that was trained on data of previous uses of the DME treatment device inside a treatment plan.

The system may include the use of the protocol and the command words of the simple device. The protocol may include commands that pre-exist in the interface for the device intended for a wired remote controller.

The machine learning trained algorithm may be trained on the gathered treatment/recovery data, treatment plan adjustments, and outcomes, and other data.

The term “simple device,” as used in this document, is referring to any device not as capable as the smart device. A simple device may also be known as a dumb device. For example, a simple device may be a DME capable of providing treatment to a patient but fails to record anything electronically about the treatment that was provided. A simple device may display treatment information to the patient but fail to store that information. A simple device may gather treatment information to display it to a user, but may not communicate (for example, transmitting wirelessly or by wire over a network to a backend server) the information electronically off the simple device.

A simple DME treatment device may be turned into a smart DME treatment device by connecting it to a smart controller.

When a surgeon performs orthopedic surgery, like a knee replacement, the surgeon may prescribe the use of a Durable Medical Equipment (DME) treatment device that provides compression, cold or heat treatment.

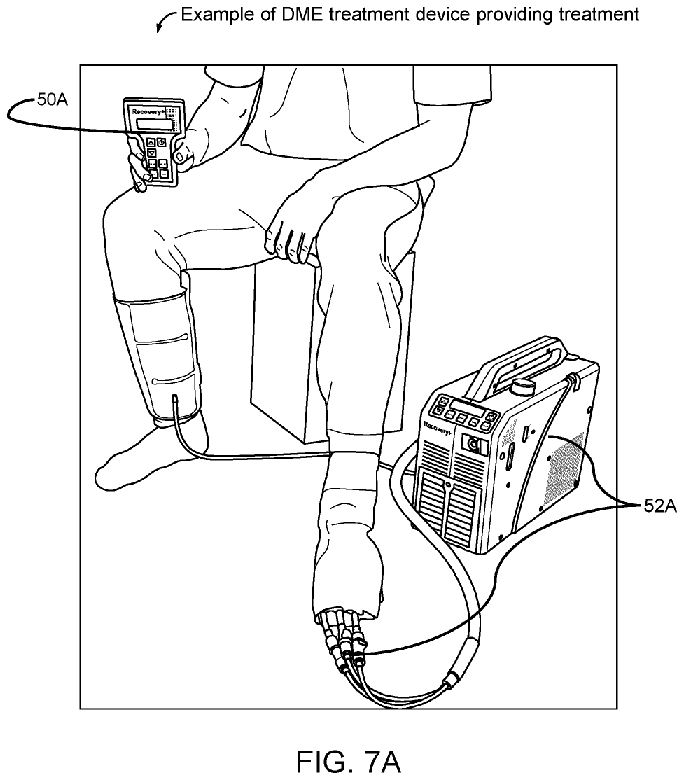

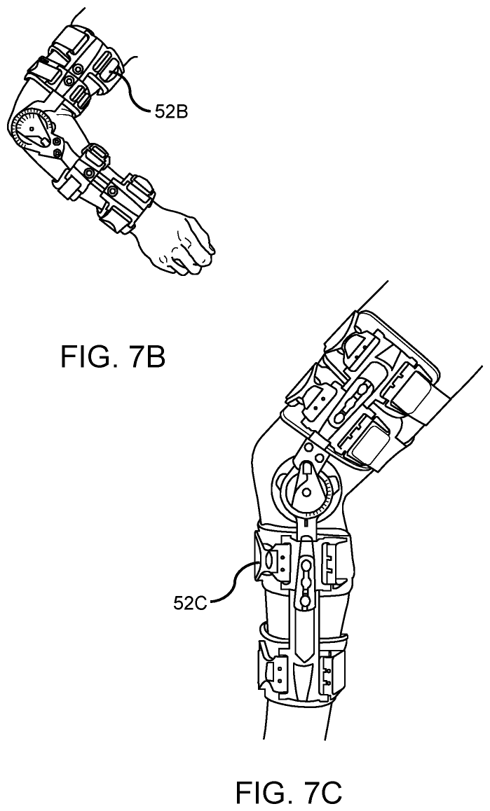

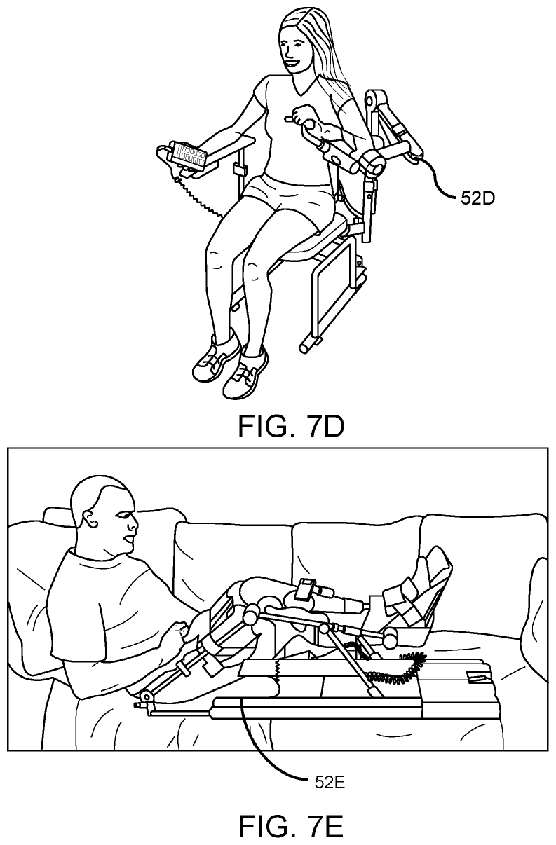

FIG. 7A, illustrates the use of thermal compression (cold and compression at the site of the surgery), also known as a DVT prophylaxis treatment. DVT prophylaxis treatment has been shown to reduce inflammation, reduce pain, and reduce the use of pain medication while increasing range of motion post-surgery.

The compression can be provided with a mechanical device (for example, calf wraps that squeeze the calves) and may be used to help prevent blood clots and prevent Deep Vein Thrombosis (DVT), that has blood clots forming in the legs and breaking away travel through the blood stream and depending on where the clot lands can block blood flow which can be very painful or deadly depending on the location.

An accelerometer may be used to monitor the movement of the patient (for example, their walking or ambulation) after surgery.

The DME treatment device 52 may be connected to a smart controller 170 (also known as a monitoring unit, an interface device or controller device) to create a smart treatment device. The smart controller 170 may report back via a communication network and cloud to a backend system.

The backend system may make the recovery data available, for example via a website or app. The system may authenticate a medical a practitioner (for example with a login and password) and authorize (for example, the login ID is recorded in the backend system as being allowed access to gathered recovery data). The recovery data may include the time the medical equipment (e.g., DME treatment device) was used (i.e., usage data), for example the length of time of the treatment session, date and time of the treatment session, and the type of treatment session (for examples what modalities of treatment was provided during the treatment session).

The backend system may do machine learning on the recovery data to suggest treatment plan adjustments.

The backend system may train a machine learning algorithm on the gathered recovery data so the algorithm can suggest a treatment plan adjustment that may lead to the best outcome. For example, an Artificial Neural Network trained on the gathered recovery data that can suggest interventions to improve the outcome for the patient.

The backend systems may be specialized to only gather data up for a particular type of surgery and a particular type of treatment device, for example, DVT prophylactic for knee surgery. The backend system may gather data up about many surgeries and treatment devices. The recovery data may be proceeded by an intervention like surgery, or may just be part of a treatment plan for a detected medical condition, for example, diabetes. The backend system may be accessed by medical practitioner.

The medical practitioner 26 may be a doctor and their staff, a group of doctors and their staff, the staff of a hospital, the staff of a group of hospitals, employees of an insurance company.

The treatment recovery data gathered may be for patients for the doctor, the group of doctors, the hospital, the group of hospitals, the insurance company, a nation or the world, or any size that proves useful for gathering the recovery data, for example, so the recovery data can be used to train a machine learning algorithm.

The medical provider access system 24 may display the treatment recovery data. The medical provider access system 24 may accept treatment plan adjustments. For example, a medical practitioner 26 may review the treatment recovery data and then may input into the system adjustments to the treatment plan.

The acceptable glucose level for the patient conditions may be set or reviewed by the medical provider, and the acceptable glucose level may be entered into the medical provider access system.

The medical provider access system 24 may have received the recovery criteria. Or the medical provider may have in their mind the recovery criteria, in which case the medical provider access system 24 would get input as to if the recovery criteria is being met, or the provider access system 24 may just receive treatment plan adjustments.

For example, the system may show problematic recovery data. Problematic recovery data may be data indicating low use of the DME smart treatment device. More specifically the usage is less than the treatment plan called for. Less usage may be for example in terms of the number of cycles (i.e., number of times used), or total usage time, or some other indicator of usage relevant to outcome success.

Problematic recovery data may be the data indicates little or minimum ambulation of the patient, for example, based on an accelerometer sensor in a range of motion brace.

Problematic recovery data may be reports of pain or swelling that may be input into the controller application running on the mobile device.

A medical practitioner on seeing this problematic treatment day, may implement an adjustment to the treatment plan to try and get the treatment to be in-line with what is needed for a successful outcome. The adjustments to the treatment plan may include the controller App automatically alerting the patient to use the smart treatment device if a certain amount of time goes by without the smart treatment/monitoring device 16 being used. The adjustment to the treatment plan may provide reminders, for example, show on the application-screen a reminder, send a text reminder, to the patient when it is time to use the device. The adjustment to the treatment plan may have the App to provide positive reinforcement by displaying a positive message, like a “good job using the treatment device”. The adjustment to the treatment plan may have the system 10 prompt the patient if they would like reminders sent to use the smart treatment device. The adjustment to the treatment plan may have the system 10 notify the patient when the patient is not taking the actions that are indicated on the treatment plan. The adjustment to the treatment plan may have the system 10 order Physical Therapy (PT) for the patient to ensure compliance with the treatment plan. The adjustment to the treatment plan may have the system 10 provide notice and track that a medical worker (for example, a person at the doctor's office) has called and talked to the patient to come up with a plan to increase the patient's use of the smart treatment device.

The medical provider access system 24 may receive notification that there has been a prescription for anticoagulants; the notification may be displayed on the controller App. An anticoagulant prescription may be particularly relevant when the patient is at risk because they are not moving (for example, ambulating) enough. Or the prescription may be issued because the recovery data indicates low or no use of the mechanical DVT prophylaxis (i.e., the smart treatment/monitoring device).

The smart device remote controller App 110 may receive and display an indication that additional interventions have been ordered, for example, that the help of home health providers has been ordered.

The medical provider access system 24 may show good recovery data. Good recovery data may show consistent use of the DME smart treatment device that equals or exceeds the treatment plan. Good recovery data may be ambulation at or above the treatment plan.

Motion or ambulation may be determined by various sensor or input, for example from an accelerometer on the smart treatment/monitoring device, mobile phone movement data (for example accelerometer data) or changes in the cell phone reported location over time.

Good recovery data may be from the smart remote controller App 10, which the patient uses to report little pain and swelling.

A medical practitioner, on seeing this good treatment day, may make a treatment plan adjustment. The Treatment plan adjustments may move the patient to the next phase of recovery.

The treatment plan adjustment may have the patient go back to work/school/life with follow up 90 days unless condition changes. During the 90 days, the treatment plan may continue the current use of the smart treatment device 16 and ambulation order.

As described above, based on the use of the smart treatment device 16 and ambulation, two significantly different treatment plan adjustments may be provided.

There may be factors to consider in a treatment plan that may include, for example, oximeter readings, glucose, weight, etc. that may be considered when deciding on a treatment plan adjustment.

A treatment plan is criteria, stored in the system 10, that recovery data can be checked against to see if the patient is performing activities known to lead to good outcomes. The treatment plan may also be known as a post-surgical recovery plan, a plan of care, a recovery plan, care plan, etc.

The treatment plan may start as the standard plan provided to the patient on discharge. A treatment plan adjustment, once implemented, becomes the new treatment plan, or current treatment plan or just treatment plan.

The system 10 may know the treatment plan and treatment plan history, The system 10 may include conditions or parameters. Alternately, the treatment plan may be generated by the backend system from a Machine Learning trained on the previous collected treatment/recovery data and outcomes.

“Outcomes” are final designation of the success or failure of the intervention, including time and effort to achieve the final designation.

Treatment plan adjustment may be suggested by a machine learning algorithm to the medical practitioner in the medical provider access system 24.

The treatment plan adjustments may be provided directly from the system 10 by a treatment suggestion algorithm and that treatment suggestion algorithm may be an Al machine learning derived algorithm.

The DME smart treatment device 16 may be any piece of equipment that can provide treatment. The DME smart treatment device may monitor directly or indirectly data about the patient 14. The DME smart treatment devices may be a medical device that monitors a physiological parameter of a patient, for example heart rate, blood pressure, body temperature, glucose levels or other parameters dealing with the functioning of the body. The monitoring may be accomplished by direct contact with the patient, with line of sight to the patient or some other way of getting a signal from the patient's body.

The patient 14 may be under the treatment of different types of doctors. The Patient 14 may be under the care of an Endocrinologist (studied in hormones of the body). The patient 14 may be a post-surgical patient, under the care of his or her surgeon or a physiatrist (rehabilitation doctor). The patient 14 may have received surgery and the treatment plan may be the post-surgical recovery plan. The patient 14 may be under the care of an endocrinologist for diabetes before undergoing surgery.

In this situation the patient may have multiple parameters being gathered in the treatment/recovery data.

The recovery data may include multiple parameters, for example, post-surgical parameters like the DVT Prophylaxis usage and glucose level.

The system 10 may include the DME smart device “equipment tracking” where the system records when the DME smart device was delivered, its current location, when it was picked up, etc.

The DME smart treatment device 16 may include oximeters, glucose monitors, weight scale, CPM (Continuous Passive Motion) devices, DVT Prophylaxis devices, etc.

In the case of a post-surgical patient that also has other comorbidities, the medical provider or the machine learning trained algorithm may specify compliance criteria that the system 10 is looking at and the system 10 may calculate an overall patient compliance score.

Recovery data may include various gathered treatment parameters from one or more DME smart treatment/monitoring devices 16.

The system 10 may have for a gathered recovery data parameter a compliance criteria. The compliance criteria may be a specific threshold number (steps taken, glucose levels, minutes of use of mechanical prophylaxis) that is a threshold to exceed. The compliance criteria may be a specific range that the treatment parameter should remain within. The system may look for a number outside the compliance criteria, and when encountered, then the system 10 may make a suggestion in the medical provider access system 24 or make a treatment plan adjustment.

The system 10 may send push notifications depending on the gathered recovery data. For example, if the recovery data is within the compliance criteria, then an “all is well” message may be sent. If the gathered recovery data is outside of the compliance criteria, then an “alerts outside the normal or expected” may be sent.

The message may be sent to a communication channel associated with a medical professional assigned to monitor the recovery, or sent to a communication channel associated to the patient.

The communication channel may be, an email, a text message to a phone (for example and SMS text), a voice message to a phone, a message displayed in the remote-control App 110, etc.

The system 10 may have recovery data that is outside the compliance criteria, indicating that the patient is out of compliance, where the compliance criteria may be the range expected for a healthy recovery. The criteria may be a single criterion or a set of criterion. In that case the system 10 may send a push notification, the notification may go to the patient, the medical practitioner or other parties that can take action. In the case of an extreme situation, the system 10 may cause immediate interaction ranging from a phone call to notifying the patient or caregiver that the patient needs to head to the hospital to be admitted.

The system 10 may be used to remotely monitor and then treat the patient and monitor progress throughout the patient treatment plan. Treatment may be provided with the use of the smart DME treatment/monitoring device 16.

The treatment plan may include measuring the blood glucose level of the patient in which case the smart DME treatment/monitoring device 16 may just have the smart controller 170 that is configured to gather data from sensors, i.e. gather compliance data. For example, a glucose monitor, a weight scale and an accelerometer. The accelerometer may measure the motion of the patient to infer the amount of movement or exercise the patient is doing throughout a time period, e.g., throughout the day.

The DME smart treatment device may have various configurations, it will have the capabilities of the smart controller. The DME smart treatment device may have a DME treatment device. The DME smart treatment device may have a sensor for monitoring.

The recovery data may include diet data, for example, meal data. The meal data may be received by the remote-control App 110. The received data may be of various forms, entered text descriptions of the food, food and drink selected off a list, pictures of the food and drinks, video of the food and drinks.

The medical provider access system 24 may display the recovery data.

A medical provider may be a physician, primary care physician, endocrinologist or other doctor, or nurse, hospital staff, health insurance company employee assigned to checkup on the patient, etc.

The medical provider may view the data gathered (e.g., blood glucose, weight, diet, movement) for a specific patient. The system may generate a suggested treatment plan adjustment and present that (for example, show it on a screen). The medical provider access system 24 may receive a treatment adjustment for a particular patient. The system may generate a treatment plan adjustment and implement it.

The system may convey to the patient the treatment plan adjustment.

The system 10 may make a determination that diabetes treatment is progressing good and no adjustment to the treatment plan is necessary. The system 10 may make this determination with input from a medical provider, a Machine Learning trained algorithm, or both. The recovery data may indicate a good situation.

Monitoring data may include when insulin was taken, and thus the system may determine if the patient is using the insulin at prescribed levels (i.e. is in the compliance criteria for insulin injection) in accordance with a diabetes treatment plan.

Recovery data may include diet/meal data and the system can determine if the diet is within the guidelines (the compliance criteria for meals) of a diabetes treatment plan.

Recovery data may include weight data that is in the compliance criteria, for example indicated the patient is maintaining their weight, neither gaining nor losing weight within a criteria of some small amount of weight.

Recovery data may include glucose levels that are within the compliance criteria (within an acceptable range) for the patient conditions as set or reviewed by the medical provider. The glucose criteria may be received by the medical provider access system 24.

Recovery data may include movement data (for example, as provided by an accelerometer in the DME, or another device of the patient, i.e., their phone or movement tracker, e.g. Fitbit). From the Movement data the system may be able to determine if the movement is within the exercises compliance criteria, thus the system may conclude that the exercise or ambulation is according to the treatment plan.

Given some or all of the above indications in the recovery data of the criteria being met, the system 10 may not issue an adjustment to the treatment plan.

In a bad diabetes example, the system 10 may determine a treatment plan adjustment is necessary. The gathered recovery data may indicate that the patient is not in compliance with the treatment plan since the recovery data indicates one or more of the criteria is not being met.

The recovery data may include Glucose readings that fail to meet the recovery criteria. For example, the criteria may be an acceptable range for the glucose level and the recovery data may indicate the glucose level is not staying within the acceptable range.

The recovery data may include body weight data and the body weight may fail to meet the recovery criteria. For example, the recovery criteria may be an acceptable maximum weight and the recovery data may indicate the body weight is above the acceptable maximum weight.

The recovery data may include diet/meal data and the meal data may be either missing or fail to be within the diet/meal criteria. For example, the diet/meal criteria may be to only eat low glycemic index foods and the recovery data may indicate the consumption of high glycemic index foods like baked potatoes. Thus, the recovery data may indicate the diet is not in compliance with the diet criteria of the treatment plan.

The recovery data may include movement data and the movement data may fail to be within the exercise criteria. For example, the recovery data may indicate via fitness tracker that 8,000 steps were taken but the movement criteria may be 10,000. Thus, the recovery data may indicate the movement data is not in compliance with the movement criteria.

Given some or all of the above failures to meet the recovery criteria, the system 10 may suggest a treatment plan adjustment via the medical provider access system 24 and see if the medical provider access system 24 receives a treatment plan adjustment input by the medical provider. The system 10 may be configured to decide and communicate a treatment plan adjustment directly to a communication channel of a patient.

Treatment plan adjustment may include sending a notification to the communication channel of the patient alerting the patient about a concern that the recovery data indicates a failure to meet the criteria of the treatment plan. Treatment plan adjustment may include adjustments to the diet criteria. Treatment plan adjustment may include adjustments to the movement criteria. Treatment plan adjustment may include adjustment to the levels of prescribed medication, for example for Insulin. Treatment plan adjustment may include adding a new prescription for medication. Treatment plan adjustment may include prescription for in-home intervention for patient and diet.

Treatment plan adjustment may include sending notification to the communication channel of the medial provider, notifying them of the recovery data failing to meet the recovery criteria, indicating patient's non-compliance.

Treatment plan adjustment may include sending communication to the communication channel of the patient alerting the patient that physicians have been notified about the recovery data failing to meet the recovery criteria.

If the system 10 includes a glucose dispensing machine then Treatment plan adjustment may include adjustment to the functioning of the glucose dispensing machine.

The DME smart treatment device 16 may be Range Of Motion brace or a Continuous Passive Motion (CPV) device. The Range of Motion brace may have an accelerometer.

Post Ortho or Podiatric Surgery, the patient ambulation is critical in their recovery. By placing an accelerometer on the patient or some Durable Medical Equipment (simple DME) the smart connector may turn the simple DME into a smart treatment device. For example, post-surgery often an ambulation aid (e.g., walker/crutch/knee scooter, etc.) is used in conjunction with the range of motion brace (foot, hip, or ankle). With the ambulation aid or ROM brace having an accelerometer and a smart controller, the recovery data about the patient ambulation can be gathered as part of the recovery data. This movement data may be made available to a medical practitioner who can monitor the movement or ambulation post-surgery, for example, monitoring the number of steps/duration of ambulation; the frequency of ambulation throughout a specific time period, for example, through a day; The use of the range of motion of brace for example for the foot/knee/hip.

The system may display the gathered motion and ambulation data and recovery data from the DME treatment device (for example, cold compression, DVT Prophylaxis). The system may receive an adjustment to the treatment plan, for example, an adjustment to the therapies. The system may decide based on a machine learning algorithm trained based on previous collected recovery data, treatment plan adjustments, final outcomes and possibly other criteria found useful.

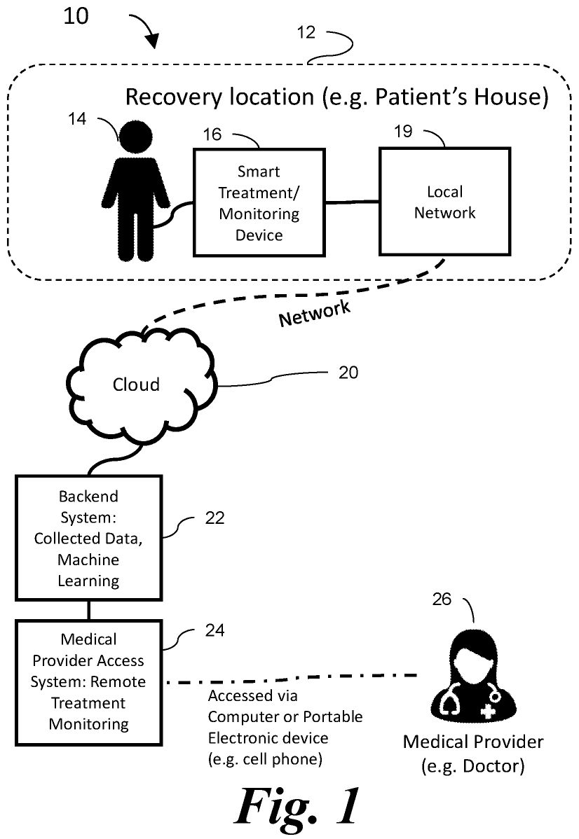

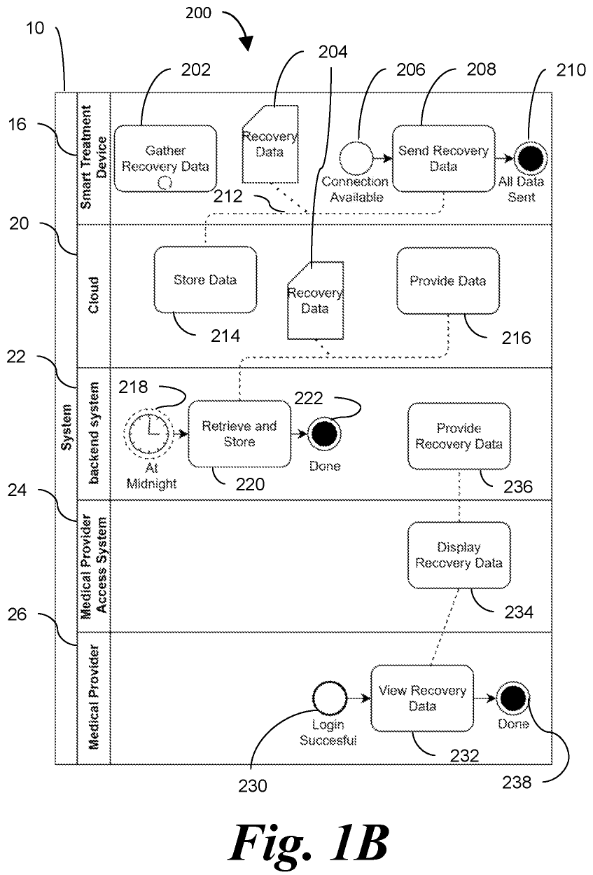

FIG. 1 shows a system 10 for gathering recovery data. The system 10 is shown with a recovery location 12. A patient 16 may receive treatment from a Durable Medical Equipment (DME) smart treatment device 16 that gathers recovery data. The recovery data may be transferred over local network 19 and the cloud 20.

The DME smart treatment device 16 may send the recovery data over to the backend system 22 (enterprise systems).

The recovery location 12 may be the house of a patient, or it may be a physical therapy clinic, or some other recovery location.

The smart treatment device 16 may include a connected mobile device. The connection to the mobile device may be a secure connection.

The DME smart treatment device 16 may include a Pulsar scientific recovery+and Deep Vein Thrombosis prophylactive treatment unit to prevent the occurrence of blood clots in the deep veins of the legs. The smart Treatment Device 16 may be provided with a smart control that gathers recovery data about the DME provided treatment and gather recovery data more generally from one or more sensors that provide physiological measurements of the patient.

The smart controller 170 may be able to create a self-forming/healing wireless connector that simplifies network formation and ensures the robust performance of the communication network.

The network 19 may be a wired network or wireless network or a combination of both. As the recovery data travels to the back-end system the recovery data may be encrypted to provide end-to-end security as it travels over the local network 19 and cloud 20.

The smart controller 170 may communicate with a DME treatment device 52 using any number of wired or wireless electronic protocols. For example communication may be over a serial port using low voltage, a parallel port, or via Universal Serial Bus (USB), or similar wired connection with customed or standard protocols. The wireless communication may include Bluetooth Low Energy (BLE), WiFi, cellular or other wireless communication protocols.

The smart controller 170 may be an add-on the treatment device 52. The smart controller 170 may connect physically to existing simple DME equipment that includes a communication port. The physical connection may provide of the existing simple DME equipment may provide power for the smart controller 170.

A smart controller 170 may also be integrated into the main control system or the smart controller 170 may be a physically separate device with separate power or be a plugin device that gets power from the simple DME treatment device.

The integration to the backend system 22 may be done through a local network 19 network cloud 20.

The network cloud 20 may include various wireless and wired computer communication networks and various communication resources. The network cloud 20 may be reached using various communication technologies and protocols, for example, Wi-Fi, cellular, and cellular bridge wireless protocols, using protocols such as HTTP, HTTPS, TLS, and may be provided by various vendors, for example, box.com, amazon web services, Microsoft Azure, Google compute engine, etc.

The smart treatment device 16 may be used by the patient 14 after surgery. The smart treatment device 16 may provide multiple treatment modalities. The smart treatment device 16 may be a monitoring device with a sensor that collects data.

The smart treatment device 16 may be a DVT prophylaxis that can provide thermal therapy, intermittent pneumatic compression at the surgical location, mechanical DVT prophylaxis (typically on each calf muscle) to prevent blood clots during the recovery phase.

The recovery data (monitoring data) may include data on any/all modalities, machine performance, component performance, and operational history, etc.. The recovery data may include the patient physiological data. The recovery data may be monitored in post-surgical situations or just in a treatment plan based on detected medical conditions or other medical situations.

Recovery data may include usage data from smart treatment device 16. The recovery data may include physiological data (physiological parameters) about the patient from sensors, and patient survey data from questions posed and answered on the control App running on the mobile device.

The network cloud 20 may be connected to a backend system 22. For example, the backend system 22 may include one or more databases for collecting data, machine learning capabilities, and remote data monitoring capabilities.

The backend system 22 may provide access to the data and machine learning capabilities via a medical provider access system 24. The medical provider access system 24 may be access with an electronic device. The electronic device may be, for example, a computer or portable electronic device, like a desktop computer, laptop, cellphone, a tablet computer, anything that can allow electronic access, typically over the internet to the medical provider 26. The medical provider 26 may access the medical provider access system to do remote data monitoring of the treatment plan.

When the smart treatment device 16 is delivered to the patient at their house, it may be delivered by a technician, the technician may instruct the patient in how to use the device, or there may be instructions on the device, instructions provided on paper, or instructions on a website or any means to enable a patient to learn how to properly use the smart treatment device 16. For example after surgery, the patient should operate the smart treatment device as instructed according to the treatment plan. The treatment device may run preloaded routines, e.g., specific programs, that perform the details of the treatment plan, where the treatment plan may be prescribed by the medical practitioner

If the medical provider wants to know if the use of the machine indicates that the treatment plan is being followed, then the medical practitioner may access the medical practitioner access system 24 to see the recovery data. The recovery data may also be continuously monitored.

The smart treatment device 16 may be used by a patient 14 with a treatment plan to deal with a detected medical condition, for example diabetics. The system 10 may gather recovery data that is from a smart treatment device 16 that lacks any active treatment but does have a sensor to gather physiological aspects of the patient, for example blood glucose measurements, or movement information from an accelerometer.

The smart treatment device 16 may provide the recovery data to the backend system after the smart treatment device 16 has been picked up. For example, the technician who is retrieving the smart treatment device 16 may have a device (for example, a cell phone) that is capable of connecting to the smart treatment device 16, taking the gathered recovery data, and sending it over the cloud 20 to the backend system 22. Alternatively, the recovery data on the smart treatment device 16 may be read when the smart treatment device has been physically delivered back to the treatment device supplier's facility, i.e., the technician's home base.

The smart controller 170 may have GPS tracking capabilities to determine the location of the smart treatment device 16. The backend system 22 may store the last known location of the smart treatment device 16. This may help prevent losing smart treatment devices 16, aid in the recovery of stolen or misplaced smart treatment devices 16. The backend system 22 may receive confirmation of delivery of the smart treatment device 16 to the patient 14 by a technician.

The smart treatment device 16 may upload the actual coordinates/address to the backend system 22. The delivery of the smart treatment device 16 to the patient 14 at a specific location may be updated in the backend system 22 without manual input of data by a technician or administrator.

The patient 14 may allow use his/her local network 19, for example, WiFi, which may be permitted the use of a wide area network (WAN) like a cellular system such as, for example, CDMA, 3G, 4G, 5G and the like, or any other wireless system. The remote-control App running on a cell phone may get the location of the cell phone and provide that to the backend systems.

The recovery data may be automatically uploaded to the backend systems, and the gathered recovery data may include specific recovery data fields. The system 10 may make the recovery data available to the medical practitioner, for example, displaying it on a webpage. The system 10 may send the recovery data to the medical practitioner, the patient, the equipment company or the insurance company. The system 10 may be configured to send the recovery data automatically or on request.

The recovery data in the smart treatment device 16 may be communicated over the network 19 and cloud 20 using various wireless modality. For example, there may have three wireless modalities in smart treatment device, for example Bluetooth, wifi and cellular.

The recovery data may be encrypted, and the communication channel may also be encrypted, for example, the communication may be transmitted through a virtual private network (VPN).

The communication of the recovery data 204 may have multiple levels of security. The system may not transmit any Personally Identifiable Information (PII). PII stands for Personal Identifiable Information and, for example, is defined by the Health Information Portability and Accountability Act (HIPPA). The system may have the ability to send recovery data 204 from the smart treatment device 16 with no PII being transmitted, for example by sending the recovery data 204 along with a device identifier. Since the device identifier fails to be connected publicly to the patient 14 no PII is transmitted.

FIG. 1B is swim lane diagram 200 with four horizontal “swim lane” showing one possible way to orchestrate activities between the smart treatment device 16, the backend system 22, the medical provider access system 24 and the medical provider 26 to allow remote data monitoring of recovery data 204.

The smart treatment device 16 swim-lane shows a gather recovery data process 202 running that gathers recovery data 204. The recovery data 204 is stored on the smart treatment device until a connection is available. At circle 206 when a network connection is available the smart treatment device 16 starts a send recovery data process 208. The send recovery data process 208 sends the recovery data 204 to the cloud 20. Once the send recovery data process 208 has sent all the recovery data 204 then the process may end at an all data sent circle 210.

The cloud 20 swim-lane shows a store data process 214 that receives the recovery data 204 sent by the send recovery data process 208, and stores the data in the cloud. The cloud 20 swim lane also shows a provide data process 216 that will provide the recovery data 204 when requested, like by the backend system 22.

The backend system 22 swim-lane shows a start timer 218 that is triggered at a specific time, for example midnight. At the specific time the start timer 218 starts a retrieve and store process 220 to retrieve recovery data 204 from the cloud 20 using the provide data process 216. The retrieve and store process 220 takes the recovery data 204 and stores it in the backend system 22. Once the retrieve and store process 220 has stored the recovery data 204 then the retrieve and store process 220 may delete the data from the cloud 20. The backend system 22 swim-lane also shows a provide recovery data process 236, that may respond to requests from the medical provider access system 24 for recovery data 204.

The medical provider 26 swim-lane shows a login successful start circle 230. When the medical provider 26 wants to view the recovery data 204 they may login to the medical provider access system 24. Once the medical provider 26 is logged in then they can enter into a view recovery data process 232 and request to see recovery data for a particular patient 14 or patients 14. The medical provider 25 may in the view recovery data process 232 request recovery data for a particular patient, for example by navigating to a view recovery data webpage on the medical provider access system 24. The view recovery data process 232 may use the display recovery data process 234 to request the data from the backend system 22 via the provide recovery data process 236. Once the medical provider access system 24 receives the requested recovery data 204 from the backend system 22 then the display recovery data process 234 may display the recovery data for the medical provider 26 to view. Once the medical provider 26 is done with viewing the recovery data then the view recovery data process 232 ends and the flow ends at the done end circle 238.

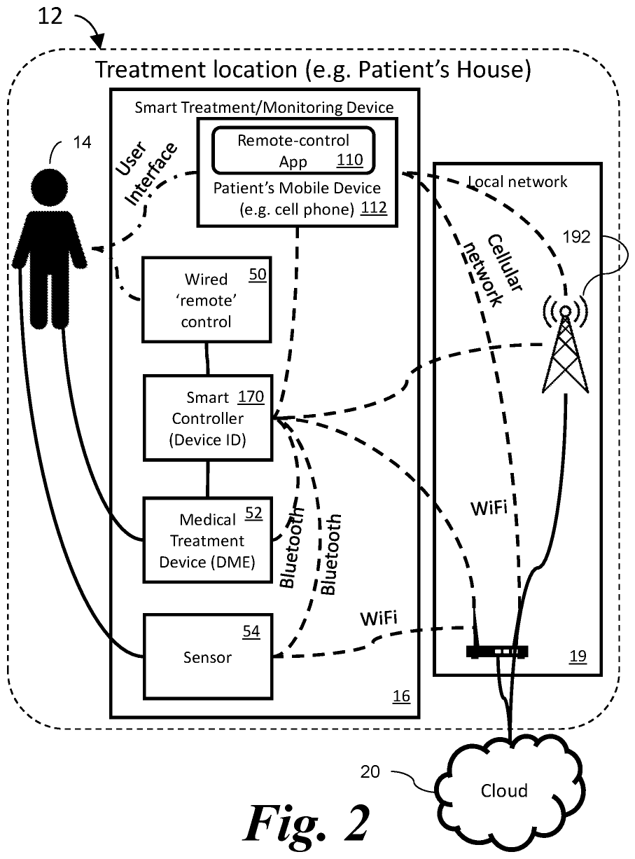

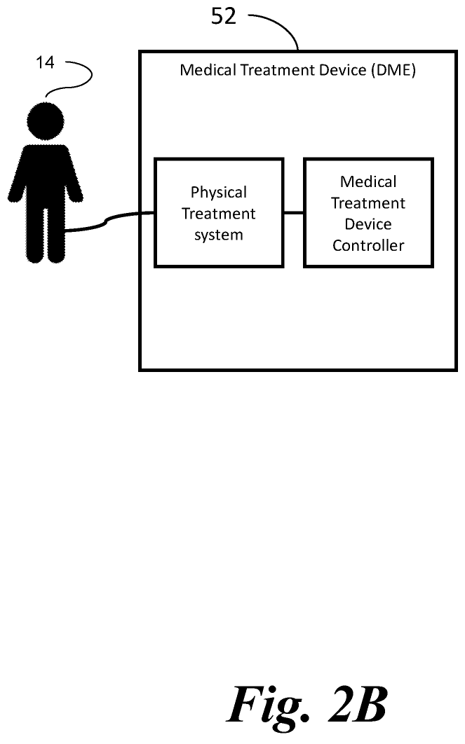

FIG. 2 is a block diagram of the recovery location 12 portion of the system 10 with details for the smart treatment device 16 and the local network 19.

The smart treatment device 16 is shown with a smart controller 170, a medical treatment device 52, a sensor 54, a wired remote controller 50, and a remote controller App 110 on a mobile device 112.

Details of the smart treatment device 16 are shown with a remote-control app 110 running on patient's electronic device 112, a wired ‘remote’ control 50. The remote-control app 110 or the wired ‘remote’ control 50 may being used by the patient 14 to communicate through the smart controller 170 to operation of the medical treatment device 52 (for example turn it on). Also shown is a sensor 54 that can provide data to the smart controller 170 for the smart controller 170 to record as part of the recovery data 204. The smart controller 170 may send the recovery data 204 over the local network 19 to be stored in the cloud 20.

The remote-control app 110 may provide a user interface for controlling the medical treatment device 52 via the smart controller 170.

The smart controller 170 may have a device ID, and the smart controller 170 may be connected (e.g., by a wire) to the medical treatment device 52. The medical treatment device 52 may be in contact with the patient to deliver treatment.

The sensor 54 may gathering data, for example, physiological data. The sensor 54 may contact the patient or may gather data about the patient without being in direct contact. The sensor 54 may transfer the data to the controller 170 via a network connection, which may be a physical connection, e.g., a wired or wireless connection, for example, a Bluetooth connection.

The smart controller 170 may receive instructions from the wired controller 50. The smart controller 170 may receive instructions from the remote-control App 110. For example, the wireless remote control 110 may be connected to smart controller 170 by Bluetooth. Furthermore, wireless remote control 110 may communicate with the cloud 20 via the local network 19.

The smart controller 170 may communicate with cloud 20 via various network technologies, for example, a cellular network, WiFi network, cellphone network or the like.

The local network 19 may be composed of various network technologies to enable the smart controller 170 to reach to cloud 20 that may include network computing resources, for example, Wi Fi router, cell phone towers, satellite receiver or other technology capable of sending data over a communication network.

FIG. 3 illustrates a block diagram of a mobile device 110. Mobile device 110 may include a control App 310, processing circuitry 320, memory 330, a GPS receiver 340 operably coupled to antenna 345, and a radio 350 operably coupled to antenna 355. Executable instructions from the memory 330 can be processed by the processor circuitry 320. The executable instructions may include instructions to run an application 310.

The controller App 310 may include: an On/Off module 368 configured to control the On/Off operation of the DME; a communication module 362 to configure the mobile device 300 to communicate with the cloud 20; a location module 364 configured to receive from GPS receiver the location of mobile device 110 and send it to cloud 20 for future use; control module 360 configured to control the DME by sending control commands via radio 350 and antenna 355. The controller App 310 may have programming stored in memory 330 run on the processing circuitry 320 to achieve the functionality described in this document.

The mobile device 110 may be a tablet, smartphone, etc. that is capable of determining the location, that way, the system 10 may have the last known location of the smart treatment device 16.

The controller App may be used to control the treatment device through the smart controller 170. For example, the controller App may enable turning on or off the treatment provided by the treatment device 52. The controller App 110 may be programmed to enable the treatment to run for a specified time. The controller App may be able to diagnose errors.

The treatment device may have one or more treatment modalities, for example, heat, cold, compression.

The control App may be downloaded onto the mobile device 110. Starting the controller App may automatically connect to the DME smart treatment device.

For example, in the case where the smart treatment device is a DVT treatment device, the DVT treatment device should be switched on before the App is started.

The control App in the mobile device 110 may be configured to provide usage instructions for startup, usage, and shutdown procedures. For example, in the case of a DVT treatment device may instruct to place wraps on calves, turn the machine on, press the start button. Then let the machine run till the time is up. Then shut the machine off, remove wraps and turn the power off.

The treatment device 52 may also be operated by a traditional wired remote control, and actions like press up the arrow button to start the unit, press power to pause, press and hold power for 2 seconds to turn off the unit. There may be a tech mode that is operated only by the technician.

The wireless communication capability for the smart controller 170 may be BLE, WiFi, Cellular, and Cellular via a bridge. The network communication in which BLE (local control) allows patients to download the App for wireless remote control. For example, the App may be downloaded on the mobile device, for example, iPhone running an Apple OS operating system or smart phone running an Android operating system.

The App running on the mobile device may use network connectivity to connect with the smart controller 170 and gather information about the treatment device 52 operations, for example, getting current machine status, and from this, the smart controller 170 may determine the usage of the machine, for example, a start time of day, how long mode of operation run. The controller 170 may determine the usage of the treatment device by the number of times per day by date and time, treatment was provided and where the treatment was provided via GPS/location.

A Machine learning module may be on the backend servers. For example, the server may be located in the cloud.

The smart controller may log, for example, the day, start time, time running. In the case of a DVT treatment, it may record the day, start time, time running, treatment mode (for example, compression, heat, or cold).

The patient may provide gather recovery data via a daily request for information through the App, and that information may be sent to the backend through the cloud. The patient data remains with the backend enterprise system, and a serial number (device ID) of the machine makes has the association to the patient protected stored security in the backend enterprise system. In this way, the system protects the patient data, so HIPAA compliance is maintained.

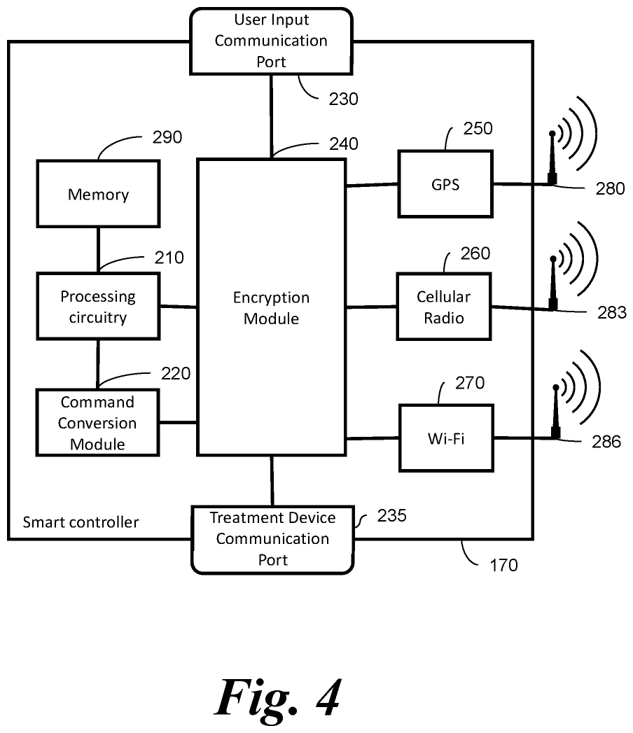

FIG. 4 shows an example block diagram of a smart controller 170. The smart controller 170 may include processing circuity 210 to process the software instructions of the smart controller 170. The processing circuitry 210 may be operably coupled to a command conversion module 220. For example, the command conversion module 220 may be configured to convert commands received that are to be sent to the DME treatment device 52 into the DME treatment device command language, structure, and protocol.

The processing circuitry 210 may be operably coupled to memory 290. For example, memory 290 may be configured to store data, location, instructions, etc.

The smart controller 170 may include a Bluetooth radio 250 operably coupled to antenna 280, a cellular radio 260 operable coupled to antenna 283, and Wi-Fi radio 270 operably coupled to antenna 286. For example, the Bluetooth radio 250, the cellular radio 260, and the Wi-Fi radio 270 may be configured to communicate with the DME treatment device 52 and the cloud. The antennas may physically be all the same antenna.

The processing circuitry 210 may be operably coupled to encryption module 240, which may encrypt the data transferred via Bluetooth radio 250, the cellular radio 260, and the Wi-Fi radio 270 and communication ports 230 and 235.

FIG. 5A shows a block diagram of a remote data monitoring (RDM) backend system 140A configured to retain the collected recovery data to enable remote data monitoring of the patient's treatment. The back end system 140A may store data, provide secure authenticated access to remote data monitoring, and capture adjustments to a medical treatment plan.

The network cloud computing 20 may send data associated with the device ID (e.g., gathered recovery data) to the backend system 140A. The system 10 at the decrypt current data loader box the data may be decrypted, and sent to the current recovery (aka current recovery data) database 502. The current database 502 may have the data associated with a particular device ID. The recovered data (i.e., recovery data) may be sent to remote data monitoring system, that is accessible via a website or App that the medical provider 26 can log into. The wired controller 50 may command the DME treatment device and a delivery tracking system that may send surgery notification, if desired. The DME treatment device and a delivery tracking system may also send notifications to the remote data monitoring system.

The backend system may have the last known location of the smart treatment device which may help prevents lost/stolen or misplaced equipment. The backend system may have gathered recovery data such as, for example, date, time of pickup and delivery including serial number (e.g., device ID), location/last known patient address and data uploaded to the proprietary cloud.

The system 10 in this document may indicate certain processes and activities taking place on specific computing components like the smart controller, mobile device, cloud, backend server etc. but in actual implementation the noted processes and activates could take place elsewhere in the system then is indicated in this document, for example converting the raw data from the Treatment device to usage information is indicated to be done on the controller, but this could instead be done on the mobile device, on the cloud, or on the backend server.

FIG. 5B shows a block diagram of a machine learning (ML) enabled backend system 140B that may provide machine learning services, data collection services and remote data monitoring.

The recovery data may be used for predictive analytics by machine learning.

The cloud 20 may send data associated with the device ID to the backend system 140B. The backend system 140B may decrypt the current data loader and send the decrypts data to DME 52. DME 52 may recover the data associated with the device ID. The recovered data may be sent to the backend remote data monitoring system,. The wired controller 50 may command the DME treatment device and a delivery tracking system that may send surgery notification if desired. The DME treatment device and a delivery tracking system may also send notifications to the remote data monitoring system.

The remote data monitoring system may send a plan of treatment outgoing adjustment to cloud 20. The remote data monitoring system may send medical data of the patient to machine learning suggestion system intervention and a database that collects the current recovery data, intervention, and outcome data. This database may send this data to historical database 145. From the historical database, the data may be sent to a machine learning module 147. The machine learning module 147 may be trained based on recovery data reading and interventions and outcomes. Based on the training, the intervention recommendation system 502 may suggest changes to the care plan when desired.

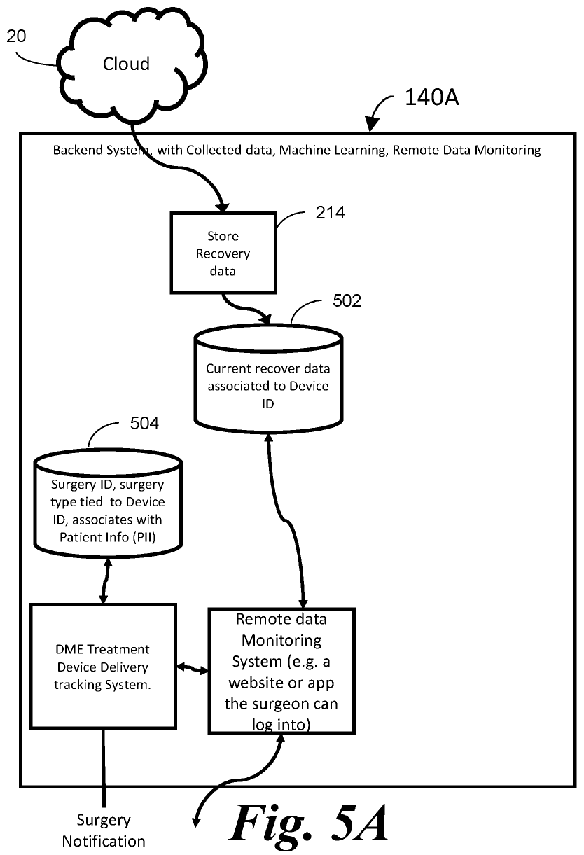

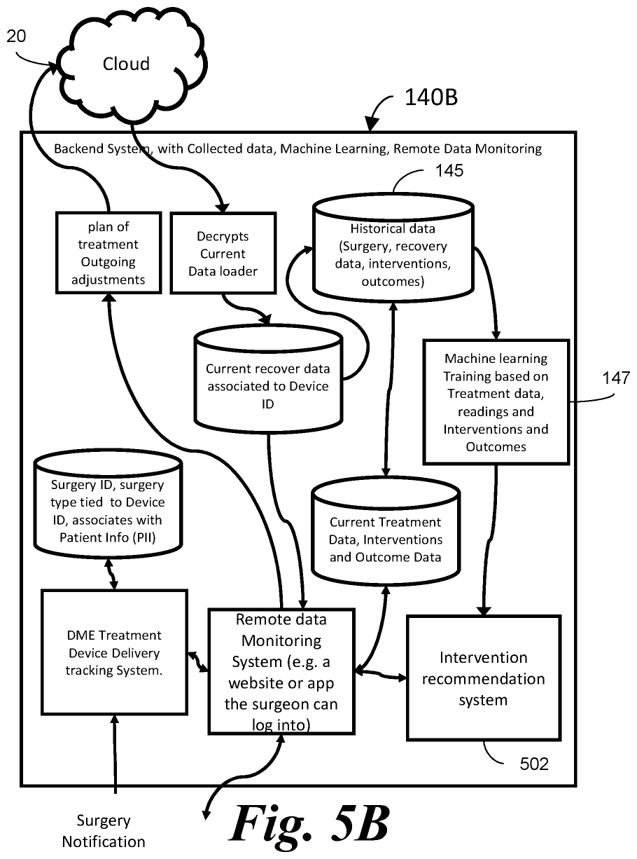

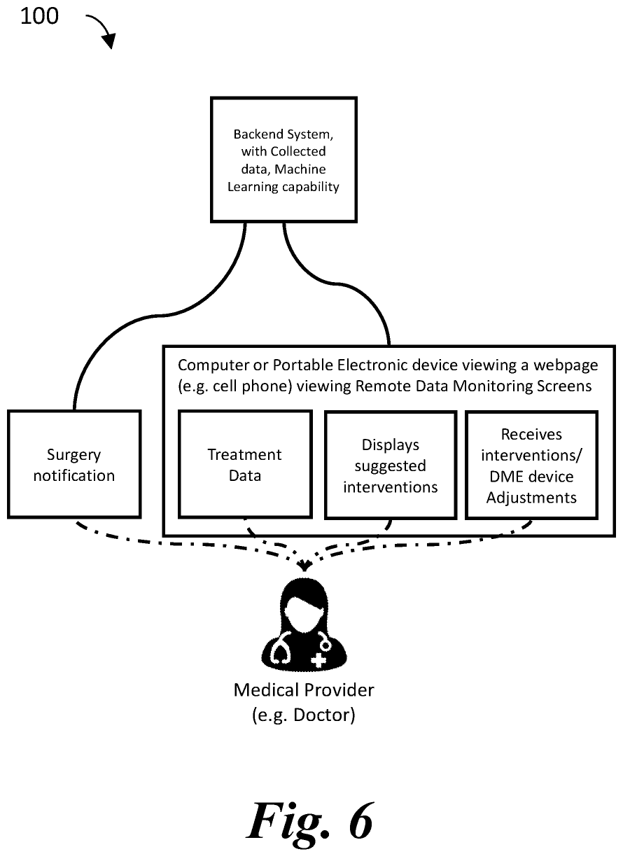

FIG. 6 shows a block diagram of a remote data monitoring system configured to provide medical data of a patient to a medical provider, in accordance with some demonstration embodiments. The remote data monitoring system is configured to provide medical data of a patient to a medical provider. For example, the system may include a backend system with collected data and machine learning capabilities. The backend system may be operably coupled to a surgery notification system, a computer or a portable electronic device, e.g., cellphone, tablet, for viewing remote data monitoring screens by a medical provider, e.g., doctor.

The computer or a portable electronic device may display recovery data, suggested interventions, and may receive interventions and may provide DME device adjustments.

FIG. 7A shows a DVT prophylactic pneumatic treatment 52A with a wired remote controller 50A.

FIG. 7B show a Range of Motion elbow limiter 52B.

FIG. 7C shows Range of Motion Knee limiter 52C.

FIG. 7D show a Continuous Passive Motion (CPM) machine 52D for the arm.

FIG. 7E shows a Continuous Passive Motion (CPM) machine for the knee 52E.

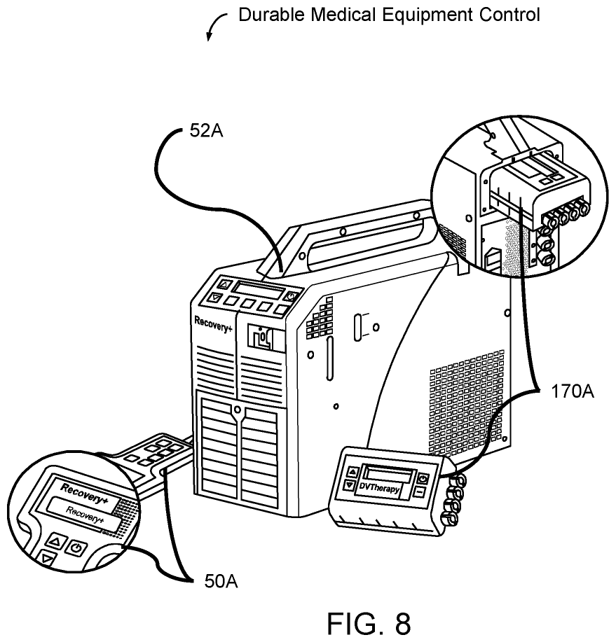

FIG. 8 shows an example of a DME control. For example, the DME treatment and monitoring system 16 may include wired control device 50A and smart controller 170A.

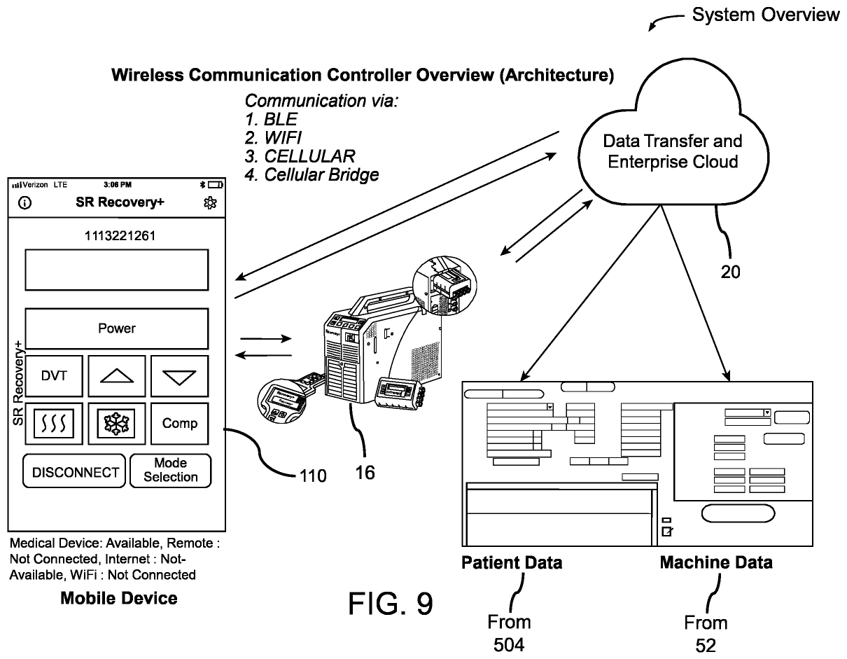

FIG. 9 shows an overview of the communication through the network cloud computing infrastructure 20 between the various components of the system 10. The system 10, as shown, includes a networking cloud 20, a DME treatment and monitoring subsystem 16, and a mobile device 110 with an App. The communication between the mobile device 110 and the DME 16 to cloud 20 may be done by using at least one of BLE, Wi-Fi, cellular, and cellular bridge wireless protocols. Cloud 20 may store patient data received from the wired control device 50 and machine data received from the DME 52.

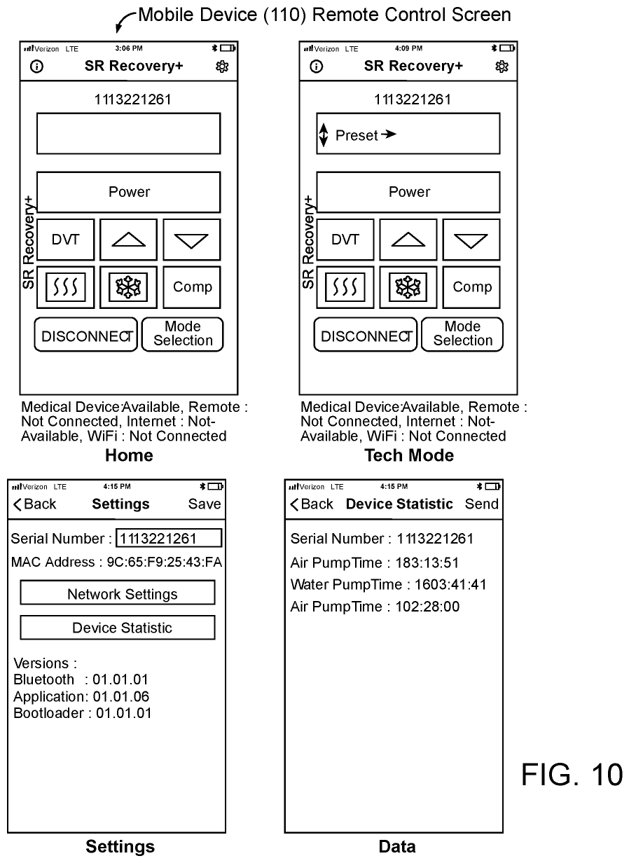

FIG. 10 shows screenshot that may be part of the remote control application specifically, a home screen, a tech mode screen, a setting screen, and a data screen.

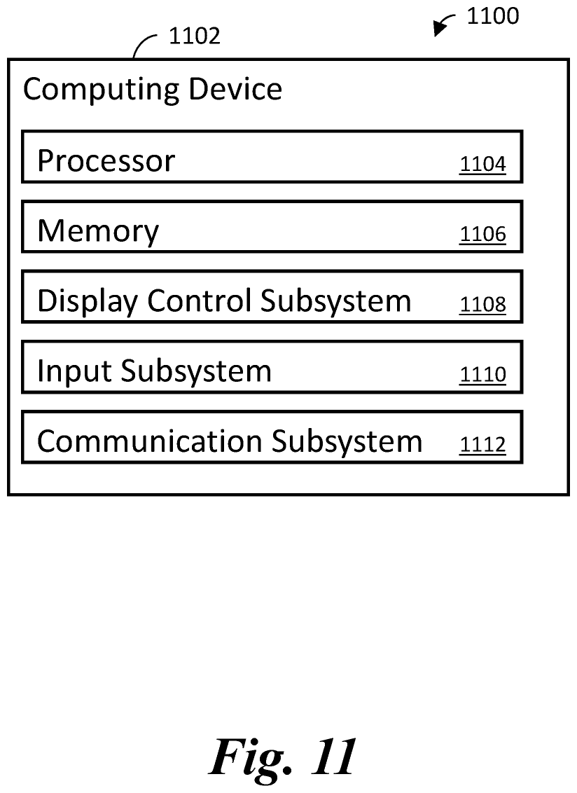

FIG. 11 is a block diagram 1100 of a computing device 1102. The device 1102 comprises a processor 1104, memory 1106, a display control subsystem 1108, an input subsystem 1110, and a communication subsystem 1112. The computing device 1102 may be incorporated into the devices described in this document and may provide the features and functionality described in this document. The processor 1104 may be configured to execute machine-readable instructions. The processor 1104 may be configured to execute the instructions on one or more virtual or physical electronic processing devices. For example, the processor 1104 executes instructions to perform the steps and processes described in this document. The processor 1104 may include one or more processors to execute the machine-readable instructions.

The memory 1106 includes a computer-readable medium that contains instructions that the processor 1104 could execute. The computer-readable medium (also referred to as a processor-readable medium) may include any non-transitory ephemeral (like not radio waves), or tangible medium that participates in providing data (e.g., instructions) that may be read by the processor 1104 of the computing device 1102. Such a medium may take many forms, including, but not limited to, non-volatile media and volatile media. Non-volatile media may include, for example, optical or magnetic disks, solid-state memory, a floppy disk, a flexible disk, a hard disk, a magnetic tape, any other magnetic medium, a CD, a CD-ROM, a DVD, any other optical medium, punch cards, paper tape, any other physical medium with patterns of holes, ROM (Read Only Memory), a PROM (Programmable Read-Only Memory), an EPROM (Erasable Programmable Read-Only Memory), a FLASH-EPROM, a USB drive (e.g. Thumb Drive), SD cards, any other memory chip or cartridge, other persistent memory, or any other medium from which a computer can read.

Volatile media may include, for example, RAM (Random Access Memory) like static random-access memory (SRAM) or dynamic random-access memory (DRAM), which typically constitutes a main memory. The memory 1106 stores information and computer-executable instructions necessary to carry out the processes described in this document. The display control subsystem 1108 facilitates displaying the media by sending signals to a display screen. The computing device 1102 provides an integrated display control subsystem 1108, memory 1106, and processor 1104 such that computing device 1102 executes the machine-readable media to provide the methods described in this document.

The input subsystem 1110 receives user input. The input subsystem 1110 connects to and receives input from devices such as a mouse, a keyboard, a touch screen, a touch screen with a keyboard, a touch screen with a number keypad, a microphone, a camera. For example, a user may indicate that the computing device 1102 is to execute a certain task, such as requesting the computing device 1102 to display any of the information described in this document.

The communication subsystem 1112 allows the execution of the methods described in this document over a network. For example, the communication subsystem 1112 enables the computing device 1102 to communicate with a plurality of other computing devices running the programs described in this document on other servers. The program may run on the other servers in parallel.

The communications subsystem 1112 is configured to receive computer instructions for the processor 1104, and those instructions are stored in the memory 1106. The communication subsystem 1112 is configured to communicate with a network by one or more transmission media, including wired (coaxial cables, copper wire and fiber optics, including the wires that comprise a system bus coupled to a processor of the computing device) or wireless.

The communication subsystem 1112 is equipped to communicate over many technologies that may be part of the network. For example, the communication subsystem 1112 could be equipped with a Wi-Fi module that connects to mobile hotspots (via WiFi) which may connect to the intemet. Wireless communication includes a cellular wireless network, Wi-Fi communications network, a wired Ethernet network, or any communication means that facilitate networked communication with other computing devices. In addition, the communication subsystem 1112 is capable of communicating via any number of short-range wireless technologies, for example, Bluetooth, Near Field Communication (NFC), ZigBee, infrared, Wide Area Network (WAN), etc. In general, the processor 1104 is configured to receive instructions, for example from the memory 1106 and executes those instructions, thereby performing the functions described in this document. Such instructions and other data are stored and transmitted using a variety of computer-readable media.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments and should in no way be construed so as to limit the claims.

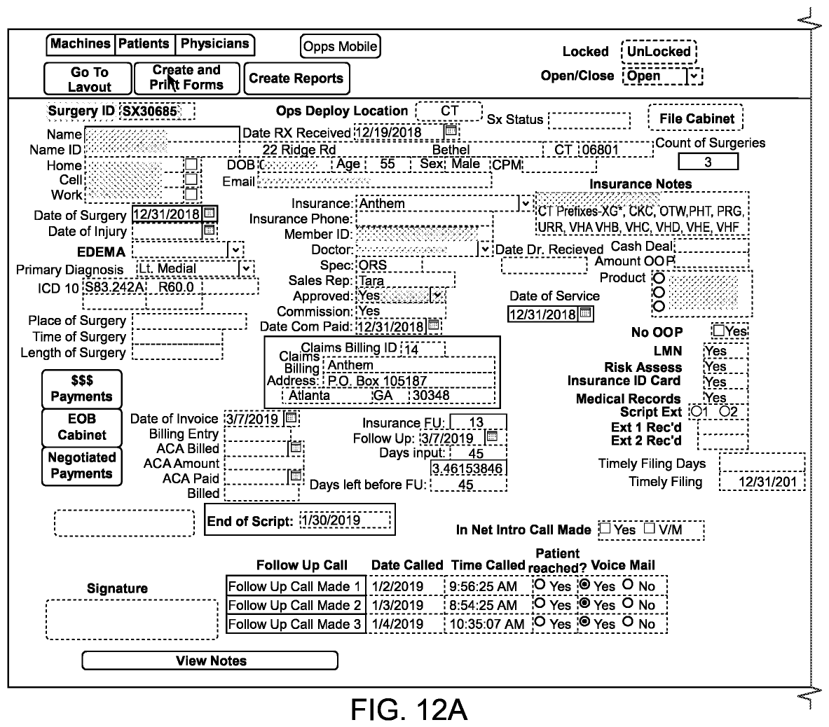

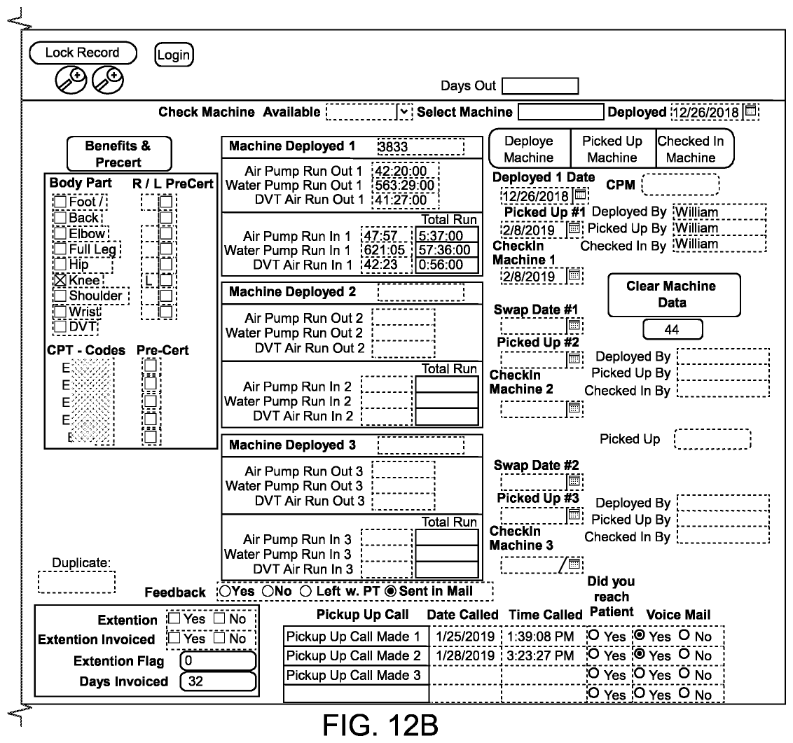

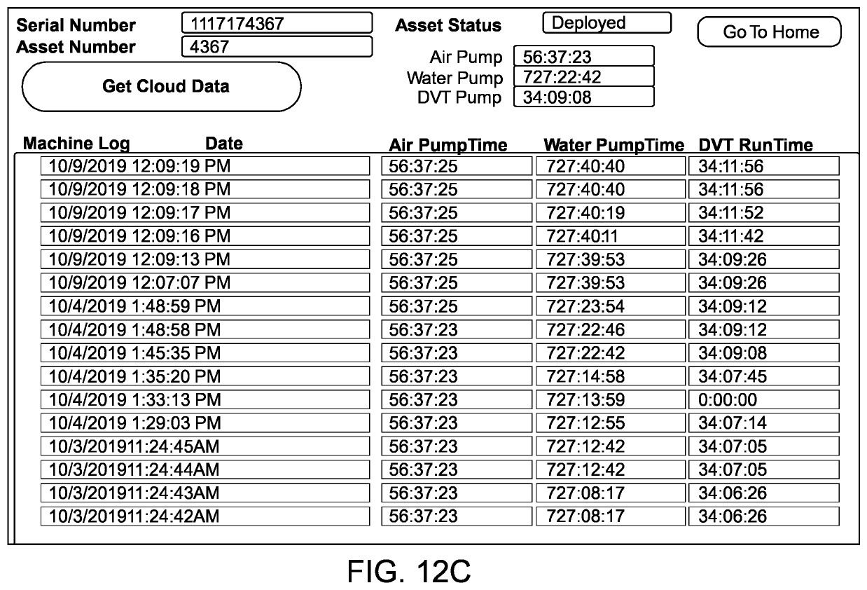

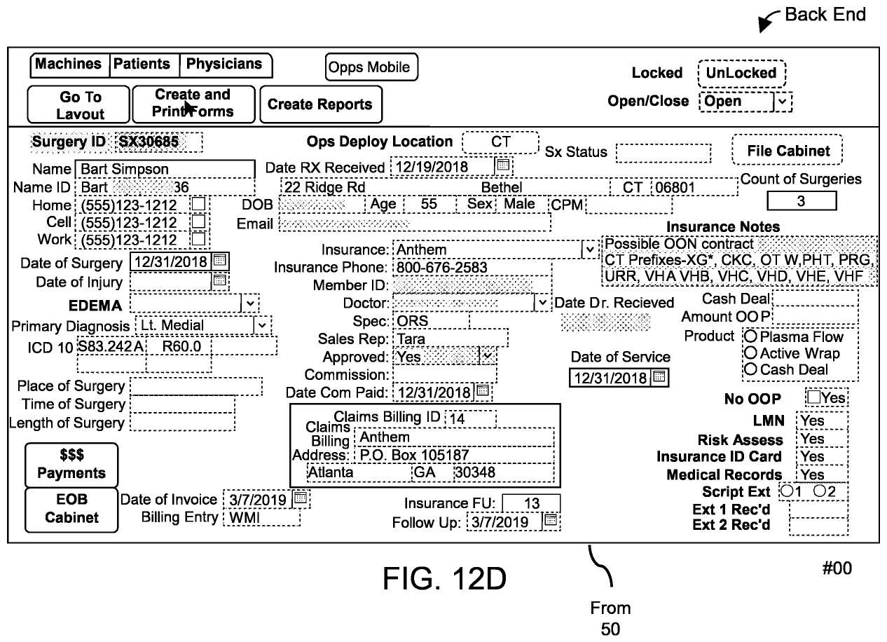

FIGS. 12A, 12B, 12C and 12D show examples of data logs of a patient, in accordance with some demonstration embodiments.

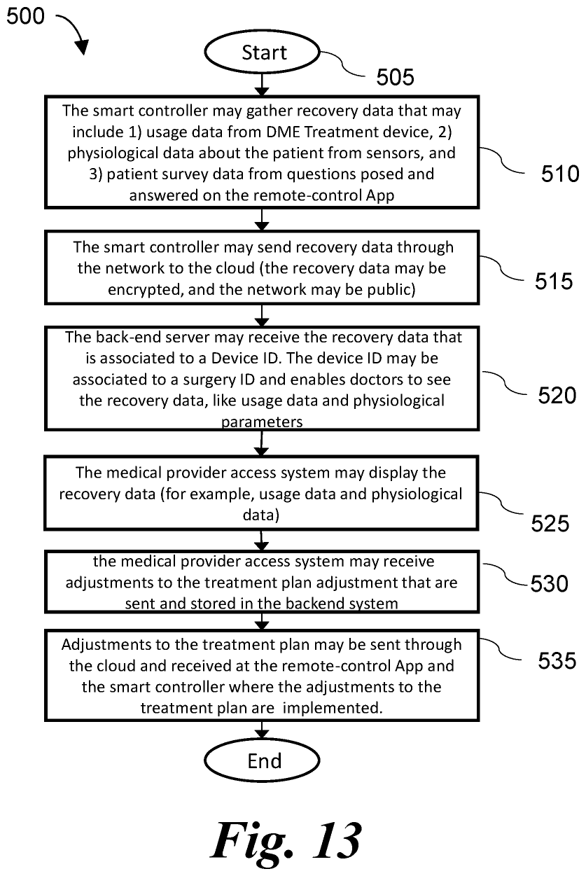

FIG. 13 illustrates a flow chart 500 showing one way the system 10 may operate.

The flow chart 500 may start at box 505.

At box 510 the smart controller 110 may gather recovery data.

Next the process flow 500 at box 515 may have the controller send the recovery data through the network to the cloud, the data may be encrypted as it travels over the network which may be public. Alternatively, the data may be plain text but just associated with a device identifier.

Next at box 520 the backend server may receive the recovery data, and the backend server may associate the recovery data with a surgery identifier based on the device identifier. The device identifier may be associated with a surgery identifier and enables doctors to see the usage data and physiological parameters.

Next at box 525 a Remote Data Monitoring site may display recovery data.

Then at box 530 the Remote Data Monitoring site may receive treatment plan adjustments that are entered, for example, into a website on the RDM site.

Then at box 535 the treatment plan adjustment may be sent through the cloud and received at the App and the smart controller and implemented. Treatment plan adjustment may also sent to the physician/practitioner.

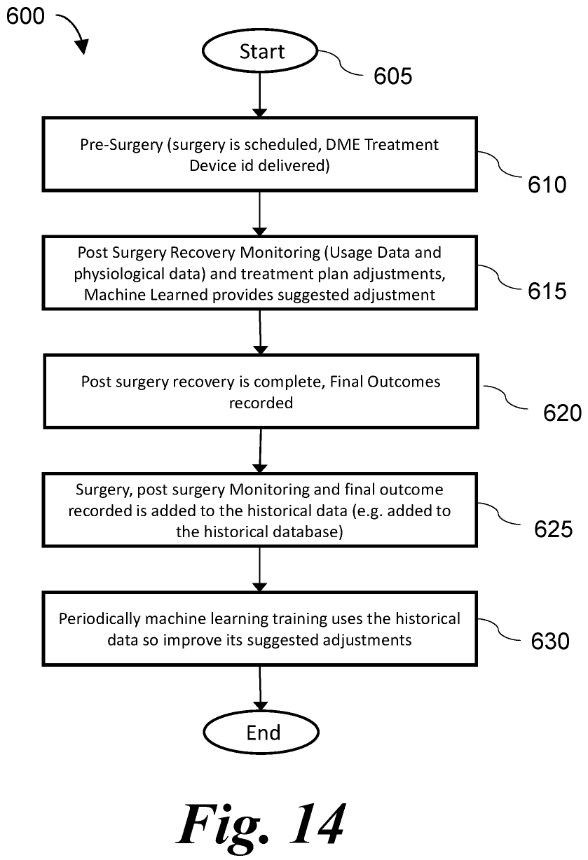

FIG. 14 shows a flow chart 600 of a method to train a machine learning module based on historical data. The flow chart 600 may start at box 605.

At box 610 the system 10 may receive pre-surgery information. For example, the Pre-Surgery stage is the stage when surgery is scheduled and DME treatment device ID delivered.

Next, at box 615, after the surgery takes place, a post-surgery monitoring (i.e., gathering recovery data) and treatment plan adjustment may be done. A machine-learning algorithm may provide a suggested adjustment.

Next, at box 620, when the post-surgery recovery is complete, the system 10 may record the final outcomes.

Next, at box 625, post-surgery monitoring (recovery data) of the surgery and the final outcomes may be added to the historical data, e.g., added to the historical database.

Next, at box 630, the system 10 may periodically have machine learning train on historical data to improve the machine learning suggested adjustment.

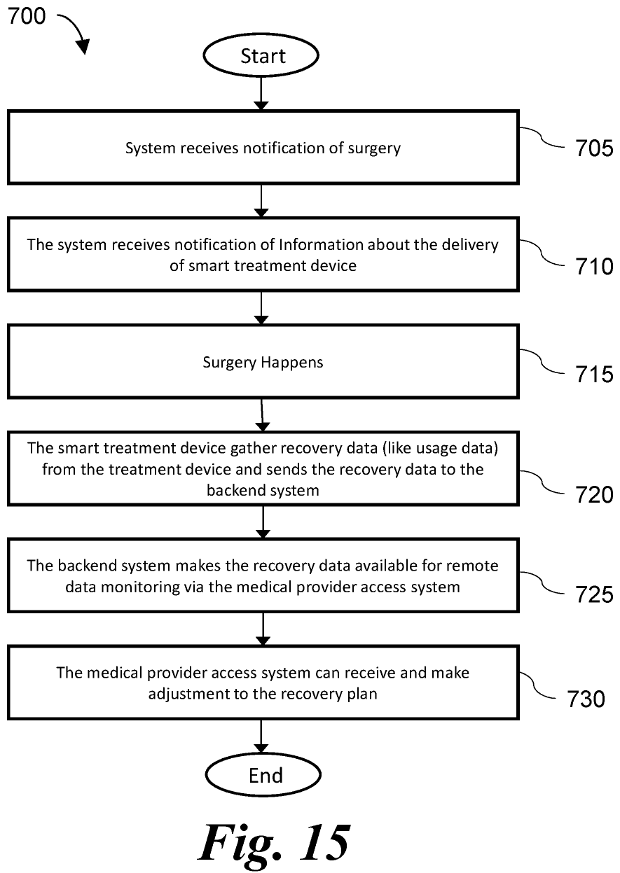

FIG. 15 shows a flow chart 700 of a method to adjust a treatment plan based on data received from DME treatment device. Flow chart 700 may start at box 705 with notification of surgery. Next at box 710, delivery of DME Treatment device with the smart controller 110 is received.

At box 715, the system determines the surgery has happened, for example because the surgery date has passed or other types of notification have been received.

Next at box 720, and after the surgery, the smart controller gathers usage data from the DME treatment device and sends the data to the backend system.

Next, at box 725, the backend system makes the recovery data available for remote data monitoring.

Next, at box 730, the system 10 can receive and make an adjustment to the plan of care (treatment plan).

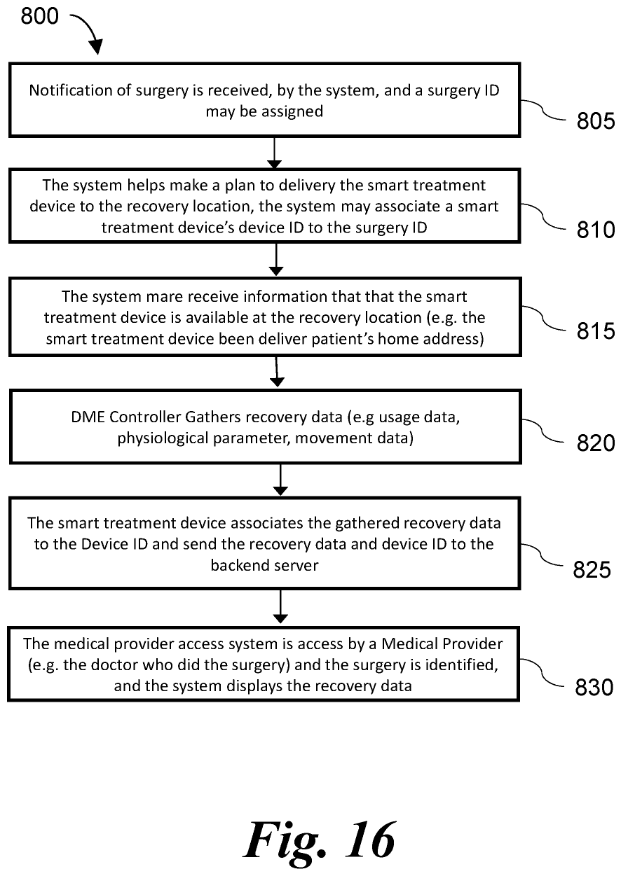

FIG. 16 shows a flow chart 800 to monitor a treatment plan based on data received from DME treatment device.

The flow chart 800 may start at box 805 when notification of surgery is received by the system 10 and a surgery identifier may be assigned.

At box 810, the system 10 helps make a plan to deliver the DME treatment device to the post-treatment, e.g., post-surgery, recovery location. The system 10 may assign a DME Device ID to the Surgery ID. The DME device ID is associated with a particular DME treatment device, for example, a serial number or asset tracking identifier.

Next, at box 815, the system 10 may record that the DME with the controller is available at the recovery location, e.g., DME has been delivered to the patient home address.

Next, at box 820, the DME smart controller may gather recovery data.

Next, at box 825, the system 10 may gather recovery data on the smart controller associate the gathered recovery data with a device ID and send the recovery data and device ID to the server.,

Next, at box 830, the system may display when authenticated and authorized access for the recovery data associated with the surgery associated through the device ID. The recovery data may be accessed by a medical provider, e.g., the doctor who did the surgery, or someone under the doctor's direction or a nurse or follow-up specialist. A medical provider may also be called a medical practitioner in this document.

The system 10 and the wireless control unit and the control application, as described in this document, are described by way of example. Other embodiments and configuration may be used, and are indeed expected to be used.

It is to be understood that like numerals in the drawings represent like elements through the figures. Not all components and steps described and illustrated by the figures are required for all embodiments or arrangements of the system 10.

It should also be understood that the embodiments, implementations, and arrangements of the systems and methods disclosed may be incorporated as a software algorithm, application, program, module, or code residing in hardware, firmware or on a computer useable medium (including software modules and browser plugins) that may be executed in a processor of a computer system or a computing device to configure the processor or other elements to perform the functions and operations described.

It should be appreciated that according to at least one embodiment, one or more computer programs, modules, or applications that, when executed, perform methods of the present invention need not reside on a single computer or processor but can be distributed in a modular fashion amongst a number of different computers or processors to implement various aspects of the systems and methods disclosed.

Thus, illustrative embodiments and arrangements of the present systems and methods provide a computer-implemented method, computer system, and computer program product for processing code(s). The flowchart and block diagrams in the Figures illustrate the architecture, functionality, and operation of possible implementations of systems, methods, and computer program products according to various embodiments and arrangements. In this regard, each block in the flowchart or block diagrams may represent a module, segment, or portion of code, which comprises one or more executable instructions for implementing the specified logical function(s).

It should also be noted that, in some alternative implementations, the functions noted in the block can occur in a different order then is presented in the figures. For example, two blocks shown in succession may, in fact, be executed substantially concurrently, or the blocks may be executed in the reverse order. Each block of the block diagrams or flowchart, and combinations of blocks in the block diagrams or flowchart illustration, may be implemented by special purpose hardware-based systems that perform the specified functions or actions, or may be implemented by the combinations of general purpose hardware and computer instructions.

The terminology used in this document is for the purpose of describing particular embodiments and fails to limit the scope of the claims. As used, the singular forms “a,” “an,” and “the” are intended to include the plural forms as well, unless the context clearly indicates otherwise. The terms “comprises” or “comprising,” specify the presence of stated features, integers, steps, operations, elements, or components, but does not preclude the presence or addition more features, integers, steps, operations, elements or components.

The phraseology and terminology used is for the purpose of description and should not be regarded as limiting. The use of “including,” “comprising,” or “having,” “containing,” “involving,” and variations thereof are meant to encompass the items listed thereafter and equivalents thereof as well as additional items.

Terms such as, for example, “processing,” “computing,” “calculating,” “determining,” “establishing,” “analyzing,” “checking,” or the like, may refer to operations or processes of a computer, a computing platform, a computing system, or another electronic computing device, that manipulate or transform data represented as physical (e.g., electronic) quantities within the computer's registers or memories into other data similarly represented as physical quantities within the computer's registers or memories or other information storage medium that may store instructions to perform operations or processes.

The terms “plurality” and “a plurality,” as used herein, include, for example, “multiple” or “two or more.” For example, “a plurality of items” includes two or more items.

References to “one embodiment,” “an embodiment,” “demonstrative embodiment,” “various embodiments,” etc., indicate that the embodiment(s) so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, unless otherwise specified, the use of the ordinal adjectives “first,” “second,” “third,” etc., to describe a common object, merely indicate that different instances of like objects are being referred to and are not intended to imply that the objects so described must be in a given sequence, either temporally, spatially, in ranking, or in any other manner.

Some embodiments may be used in conjunction with various devices and systems, for example, an Internet of things device (IoT), a User Equipment (UE), a Mobile Device (MD), a wireless station (STA), a Personal Computer (PC), a desktop computer, a mobile computer, a laptop computer, a notebook computer, a tablet computer, a server computer, a handheld computer, a handheld device, a Personal Digital Assistant (PDA) device, a handheld PDA device, an onboard device, an off-board device, a hybrid device, a vehicular device, a non-vehicular device, a mobile or portable device, a consumer device, a non-mobile or non-portable device, a wireless communication station, a wireless communication device, a wireless Access Point (AP), a wired or wireless router, a wired or wireless modem, a video device, an audio device, an audio-video (AN) device, a wired or wireless network, a wireless area network, a Wireless Video Area Network (WVAN), a Local Area Network (LAN), a Wireless LAN (WLAN), a Personal Area Network (PAN), a Wireless PAN (WPAN), and the like.

The systems and methods described may have one-way or two-way radio communication systems, cellular radiotelephone communication systems, a mobile phone, a cellular telephone, a wireless telephone, a Personal Communication Systems (PCS) device, a PDA device which incorporates a wireless communication device, a mobile or portable Global Positioning System (GPS) device, a device which incorporates a GPS receiver or transceiver or chip, a device that incorporates an RFID element or chip, or the like.

The subject matter described above is provided by way of illustration only and various modifications and changes can be made to the subject matter described without following the example embodiments and applications illustrated and described, and without departing from the spirit and scope of the present disclosure.

What is claimed is:

1. A method to improve medical outcomes implemented on an electronic computer system comprising:
receiving at a backend server notification of surgery including a recovery location,
creating a plan in the backend server to deliver a smart treatment device to the recovery location, where the smart treatment device has a unique identifier,
associating the unique identifier to the surgery,
receiving information that the smart treatment device is available at the recovery location,
gathering recovery data by the smart treatment device,
sending the recovery data with the unique identifier to a backend server,
receiving at the backend server the recovery data,
receiving a request for access to the recovery data for a surgery,
providing access to the recovery data,
receiving adjustments to a treatment plan,
implementing adjustments to the treatment plan,
receiving final medical outcome information,
recording the surgery, recovery data and final medical outcome in a historical database,
training a machine learning algorithm on the historical data,
using the machine learning algorithm to suggest adjustments the treatment plan.

2. A medical remote monitoring system comprising:
a backend server with a medical provider role with rights to access a patient identifier and that patient identifier associated recovery data;
a treatment device with a treatment modality that is performed based on commands received and record usage information about the treatment modality where the treatment device communicates the recovery data associated with a unique identifier to the backend server; and
a mobile device executing a software application configured to provide the commands, and
the backend server uses the usage information to for display over a user interface accessible having authorized access to the patient identifier.

3. The system of claim 2, where the treatment modality includes mechanical deep vein thrombosis prophylaxis.

4. The system of claim 2, where the usage information includes details related to schedule, duration, and usage of the treatment device.

5. The system of claim 2, where the recovery data includes data related to usage of the treatment device, physiological data, and data entered into the software application in response to questions presented to the patient on the software application.

6. The system of claim 2, where the unique identifier is the treatment device's identifier.

7. The system of claim 6, where the treatment device's identifier is associated with a surgery ID, and where the surgery ID is associated with the patient identifier.

8. The system of claim 2, where the treatment device is a prophylactic treatment device.

9. The system of claim 2, where the backend server provides access to the recovery data and receives interventions for improving health of the patient.

10. The system of claim 9, where the interventions are obtained from a machine learning model pre-trained on health improvement data of patients and their outcomes.

11. A method for improving treatment outcomes, the method comprising:
collecting recovery data of a patient which includes usage information obtained from a durable medical equipment treatment device, physiological data obtained from sensors, and patient entered data;
associating the recovery data with a unique identifier;
encrypting the recovery data associated with the unique identifier;
transmitting the encrypted data to a backend server;
decrypting the encrypted data at the backend server to obtain the health data and unique identifier; and
providing access of the recovery data to enable remote data monitoring by a medical provider.

12. The method of claim 11, where the recovery data is gathered using one or more of an oximeter, glucose monitor, and weight scale.

13. The method of claim 11, where the usage information includes details related to duration and usage of the treatment device.

14. The method of claim 11, where the unique identifier is the treatment device's identifier.

15. The method of claim 14, where the treatment device's identifier is associated with a surgery ID, and where the surgery ID is associated with a patient identifier.

16. The method of claim 11, further comprising storing previous patient data collected about treatment data, adjustment to a treatment plan for the patient, and final outcomes for the patient.

17. The method of claim 16, further comprising training a machine learning model on the previous patient data collected about treatment data, adjustment to a treatment plan for the patient, and final outcomes for the patient.

18. The method of claim 17, further comprising receiving a suggested adjustment to the treatment plan from the machine learning model.
